(19) United States
(12) Patent Application Publication
(10) Pub. No.: US 2025/0283027 A1
(43) Pub. Date: Sep. 11, 2025

(54) AI MANIPULATED AUTOMATED CELL CULTURING SYSTEM AND METHOD

(71) Applicant: MitoAI Inc., Pittsburgh, PA (US)

(72) Inventors: Bingda Li, Pittsburgh, PA (US); Xinyu Wang, Pittsburgh, PA (US); Jingkun Liu, Pittsburgh, PA (US)

(21) Appl. No.: 19/074,039

(22) Filed: Mar. 7, 2025

Related U.S. Application Data
(60) Provisional application No. 63/563,132, filed on Mar. 8, 2024.

(51) Int. Cl. C12M 1/36; C12M 1/00; C12M 1/12; C12M 1/26; C12M 1/34

(52) U.S. Cl. CPC C12M 41/48; C12M 29/06; C12M 33/04; C12M 37/02; C12M 41/30

(57) ABSTRACT

The present invention relates to an integrated system for automating cell culture processes. The system combines artificial intelligence (AI) with a robotic apparatus to execute tasks typically performed manually in cell culture laboratories. The AI module employs machine learning algorithms trained on extensive datasets, enabling it to make informed decisions regarding cell culture conditions and protocols for a variety of cell types. An accompanying robotic system performs liquid handling tasks such as media changes and cell passaging with precision. The system is compatible with multiple types of cell culture vessels, facilitating bulk processing. Additionally, an enclosed sterile environment is maintained to prevent contamination. A user interface allows for the customization of protocols and remote monitoring, enhancing operational efficiency. This invention streamlines cell culture workflows, reduces manual labor, and increases the reproducibility and scalability of cell culture.

### PRIORITY STATEMENT

This application is a continuation of U.S. Provisional Pat. App. No. 63/563,132, entitled “AI Manipulated Automated Cell Culturing System and Method,” filed Mar. 8, 2024. Hereby fully incorporated by reference.

### BACKGROUND

Cell culture has undergone significant evolution since the early twentieth century, transitioning from a rudimentary observational science reliant on microscopes to a sophisticated technology integral to both academic and industrial domains. This transformation has been particularly propelled by recent advancements in cell therapies and tissue engineering, which are pivotal to the expanding field of regenerative medicine focused on repairing, replacing, and regenerating tissues and organs. Cell-based treatments often demonstrate superior therapeutic efficacy compared to conventional pharmaceutical drugs and medical devices, thereby underscoring the indispensable role of cell culture in contemporary biomedical research and applications.

Presently, the capacity to culture diverse human tissue cells ex vivo is fundamental to regenerative medicine research, where the necessity for large-scale cell culture is escalating rapidly. Traditionally, cell culture in research environments is manually executed by personnel such as students, technicians, and research associates. Nonetheless, manual cell culture is encumbered by several limitations. Primarily, the elevated risk of contamination attributable to human intervention engenders costly setbacks and research delays. Additionally, the process is predicated on subjective human assessment of cell confluency and health, culminating in variability contingent upon individual expertise. Moreover, the repetitive and physically taxing nature of manual cell culture, particularly involving extensive pipetting and manipulation of cell culture vessels, can precipitate stress injuries and consume substantial time. Lastly, the inefficiency and labor-intensive characteristics of manual cell culture impede scalability, rendering it arduous to generate the copious quantities of cells requisite for specific research applications, such as 3D bioprinted organs necessitating billions of cells.

In addressing these challenges, partially automated bioreactor systems have been developed, chiefly for high-density cultures of single cell types, featuring automated regulation of medium flow, oxygen, and temperature. However, these systems still necessitate manual setup, monitoring, and harvesting, and lack full adaptability to the diverse requirements of various cell types. Furthermore, existing automated systems, including liquid handling robots and specialized cell culture workstations, provide a measure of automation but are frequently constrained in scope. They generally function under pre-programmed protocols that demand expert configuration and are devoid of real-time adaptive decision-making capabilities. The integration of multiple devices-such as incubators, imaging microscopes, and shakers-into a unified system persists as a formidable challenge, as does the maintenance of sterility and the accommodation of diverse vessel types, encompassing flasks and multi-well plates.

### BRIEF DESCRIPTION OF THE DRAWINGS

FIG. 1A depicts an isometric view of a full system (100), illustrating a two-compartment design with the cell culture incubation compartment (190) positioned below and a liquid handling compartment (105) positioned above;

FIG. 1B depicts an interior view of the full system (100), detailing the integration of key modules such as the transportation system (165), gripper (175), and user interface panel (150);

FIG. 2A depicts various embodiments of the full system;

FIG. 2B depicts the Liquid Handling Robotic System (130) as a standalone module, designed for installation within a commercial biosafety cabinet or laminar flow hood (200) to provide an enclosed, sterile environment;

FIG. 2C depicts the Liquid Handling Compartment (105) integrated into an enclosed system (205) that provides a laminar flow environment, with the compartment positioned atop an incubation system (210) featuring an automatic door mechanism. A SCARA (Selective Compliance Articulated Robot Arm) (215) is employed to transport cell culture vessels between the liquid handling compartment and the incubation compartment (190);

FIG. 2D depicts an alternative embodiment where the liquid handling compartment (105) is directly positioned on top of the automated cell culture incubation compartment (190);

FIG. 3A depicts a typical cell culture process employing the automated system;

FIG. 3B depicts the inverted microscopic imaging system (300) used for capturing cell images, including components such as light sources (305), a microscope system (310) with multiple magnification options, and an autofocus motor (320);

FIG. 3C depicts a demonstration of cell confluency analysis via the imaging system;

FIG. 3D depicts a side view of the inverted microscopic imaging system (300);

FIG. 4A depicts interior features of the Liquid Handling Robotic System (130);

FIG. 4B depicts an alternative design incorporating a horizontal gantry (420) and a side gantry (430), where a linear actuator (250) moves the Automatic Liquid Pipettor (125) coupled with a pipette (410) for liquid handling on a cell culture vessel (140);

FIG. 4C depicts a top view of the Liquid Handling Robotic System (130), highlighting the Liquid Waste Container (445) used for disposing of waste media and other fluids;

FIG. 4D depicts a front view of the Liquid Handling Robotic System (130);

FIG. 5A depicts a user interface for setting desired cell confluency levels and configuring the number of cell culture vessels;

FIG. 5B depicts a secondary view of the user interface, illustrating visualization of cells at different magnifications;

FIG. 5C depicts a typical language processing workflow for robotic system control based on user input;

FIG. 6A depicts the Liquid Supply System (110), comprising a refrigerator (600) that houses bottles (610) containing cell culture reagents, a peristaltic pump (620) that transfers liquids to a liquid heating system (120), a temporary reservoir (615) for storing warmed liquids, and quick connectors (630) for rapid tube replacement;

FIG. 6B depicts a side view of the liquid supply system (110);

FIG. 7A depicts the platform in its outer position, where liquid addition or removal operations are performed;

FIG. 7B depicts the platform in its inner position, where a linear actuator (250) with a cap adapter (255) engages the cap of a conical tube;

FIG. 7C depicts a side view of the Conical Tube Operation Platform;

FIG. 8A depicts a detailed embodiment wherein a cap opener (135), driven by a linear actuator (250), moves a cap adapter (255) to securely engage and remove the cap from a cell culture vessel (140);

FIG. 8B depicts a side view of the Vessel Operation Platform (145), showing the vessel holder (260) and integrated heating module (265) for maintaining the vessel at approximately 37° C.;

FIG. 8C depicts a side view of a cell culture vessel (140) being rotated into an upright position by the Vessel Tilting System (405), positioning the vessel for efficient pipetting by the pipette (410);

FIG. 8D depicts the Heating Module (265) on the Vessel Operation Platform (145); and

FIG. 8E depicts the Vessel Adapter (270) on the Vessel Operation Platform (145).

### DETAILED DESCRIPTION

The disclosed technology provides a fully automated, versatile cell culture system designed to support a wide range of protocols, including multi-passage cultures. The system is engineered to facilitate the adaptation of established manual protocols while incorporating modular components to enable customizable setups based on specific research or industrial applications. Additionally, embodiments may incorporate specialized tools such as microscopes and centrifuges, ensuring a comprehensive and fully automated cell culture solution.

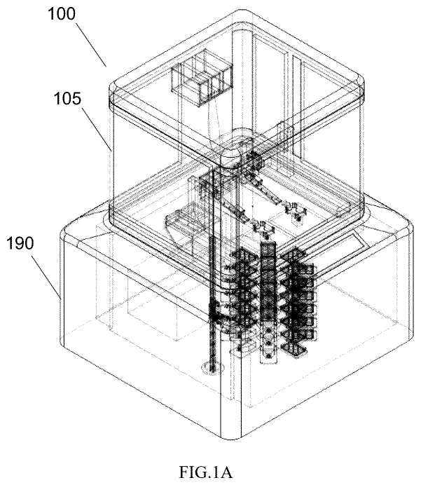

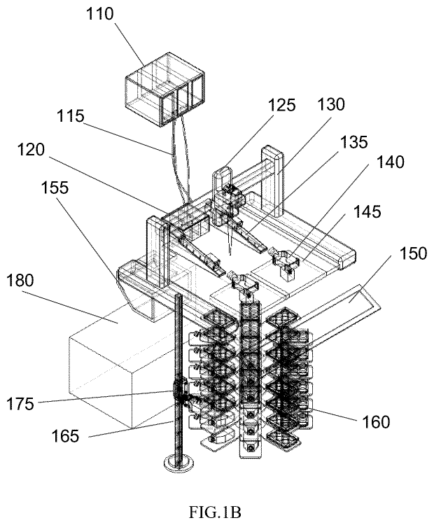

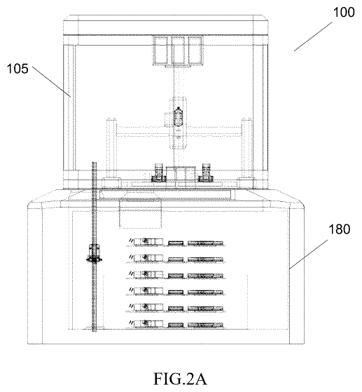

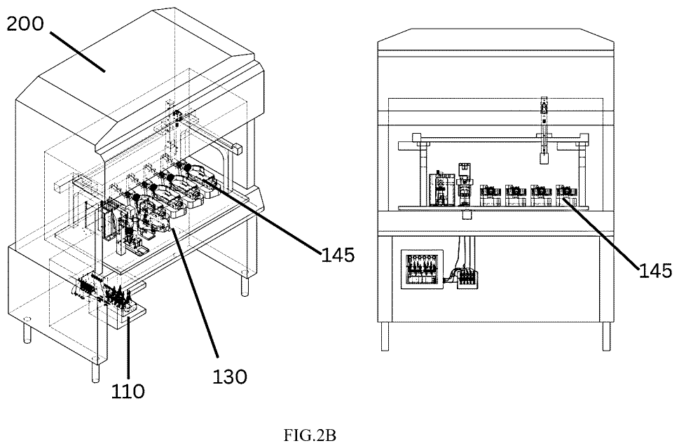

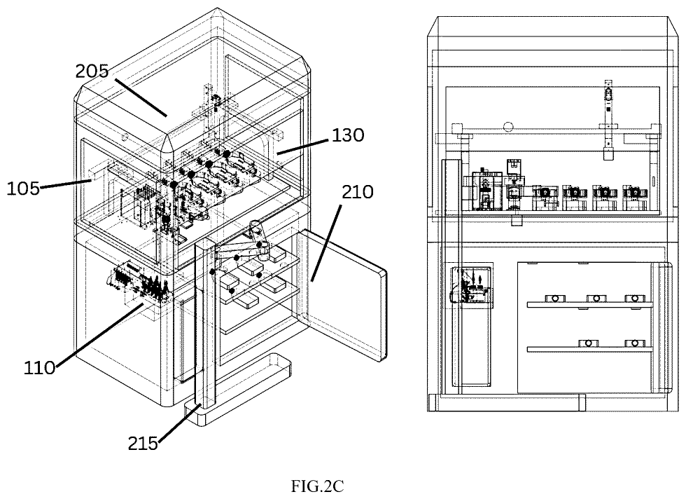

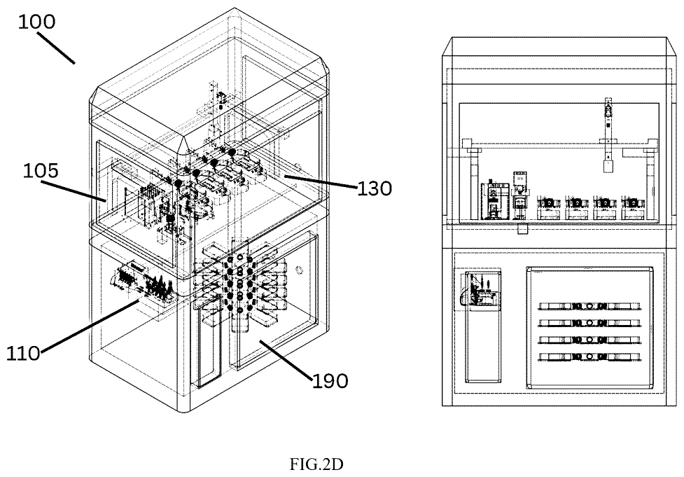

Turning now to the figures, FIG. 1A and FIG. 2A illustrate an AI-manipulated automated cell culture full system (100) in different configurations. This system consists of two primary compartments:

A cell culture incubation compartment (190), responsible for maintaining optimal environmental conditions for cell growth.

A liquid handling compartment (105), housing robotic subsystems that execute automated liquid handling operations such as media exchange, reagent delivery, and cell passaging.

The full system (100) is designed as a closed system, ensuring a sterile environment to minimize contamination risks. The incubation compartment (190) is positioned beneath the liquid handling compartment (105). When liquid handling operations are required, cell culture vessels are automatically transported from the incubation compartment to the liquid handling compartment via an integrated transportation system (165), which utilizes a gripper (175) for vessel manipulation, as shown in FIG. 1B.

In the liquid handling compartment (105), as shown in FIG. 1B, a liquid supply system (110) is included, which contains several storage containers for cell culture reagents such as cell culture media, trypsin, PBS buffer, etc. The number of containers can be modulated to fit the needs of the user. This liquid supply system functions like a mini-fridge, consistently maintaining the liquids at 4° C. This liquid supply system may deliver the reagents to a liquid heating system (120) via a tubing system (115), where the liquids are heated to 37° C. for cell culture use. In the liquid heating system, the liquids are kept in separate channels, ready for use by the robotic system. The number of channels can be modulated based on the needs of the user.

As shown in FIG. 1B, the automatic liquid pipettor (125) utilizes each reagent following the cell culture protocol. The pipette tips are discarded and replaced before coming into contact with a different liquid. The number of automatic liquid pipettor (125) can be adjusted to increase liquid handling throughput.

As shown in FIG. 1B, the waste storage (155) is located beneath the liquid handling compartment. It consists of two bins: a liquid waste bin and a solid waste bin. The liquid waste bin is used to store liquid waste, such as used media and buffers from cell culturing, which is delivered by the liquid pipettor. The solid waste bin is for storing used pipette tips. A hole at the bottom surface of the liquid handling compartment allows the pipette tips to be dropped into the solid waste bin.

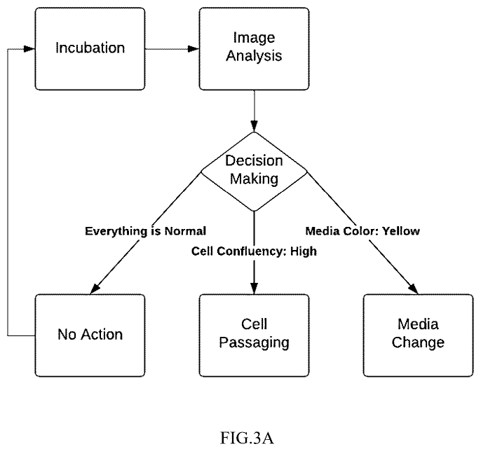

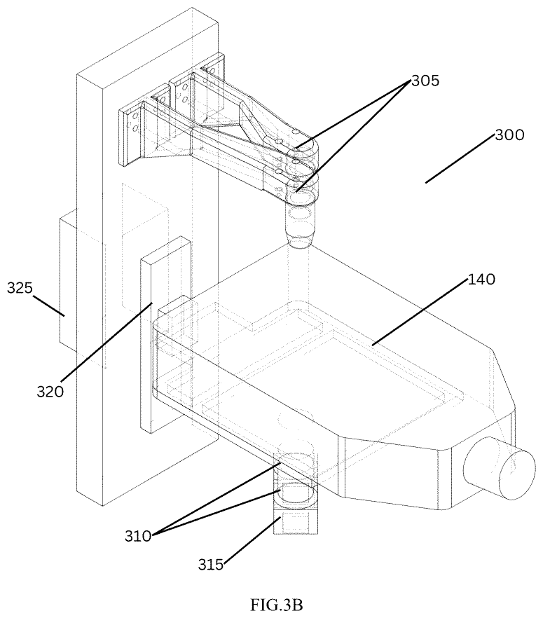

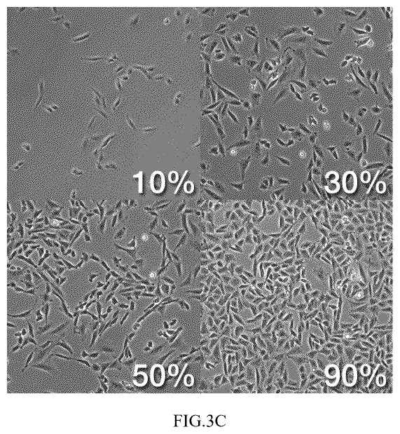

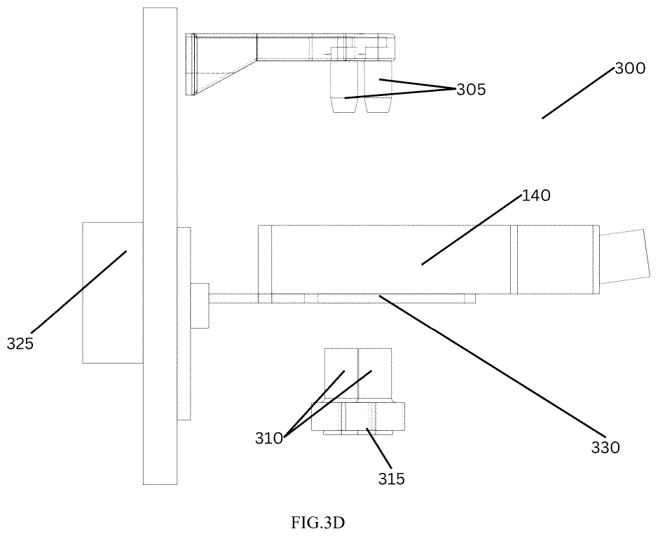

The imaging system (300), as illustrated in FIG. 3B, comprises an inverted microscope including a light source (305) and a microscope system (310) with at least one of the (4X), (10X), (20X), and (40X) magnification capabilities. This imaging system is designed to be compatible with the full system (100). It can perform auto-focus for image capturing and lens switching for different magnifications. The camera (315) captures the image reflected by the microscope. The captured image is then transmitted to the microscope computer (325) for analysis. The AI, based on this analysis, determines whether action is necessary, which may include tasks such as media changes and cell passaging. Should action be required, the cell culture vessel (140) is conveyed by the transportation system (165) to the liquid handling robotic system (130), as shown in FIG. 1B. If no action is necessary, the cell culture vessel remains in the incubator for continued culturing.

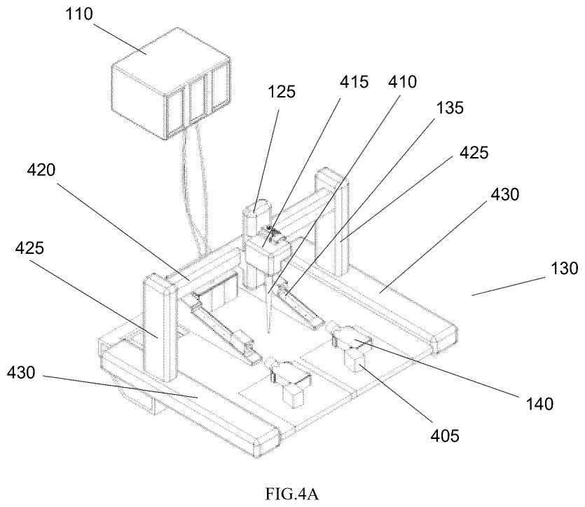

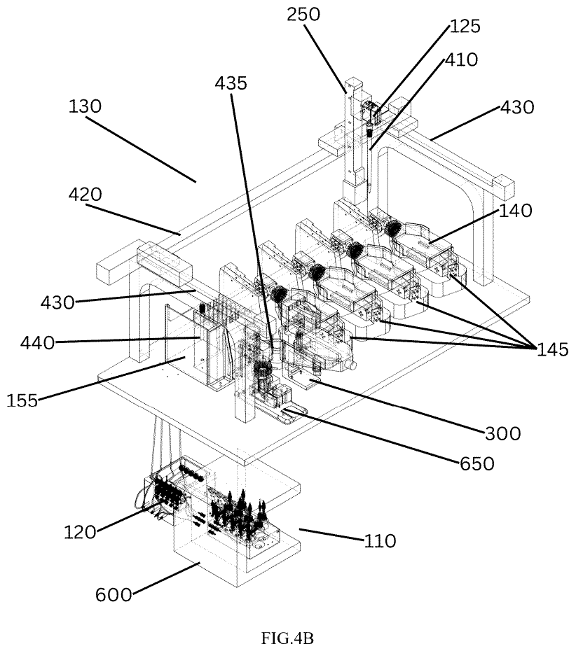

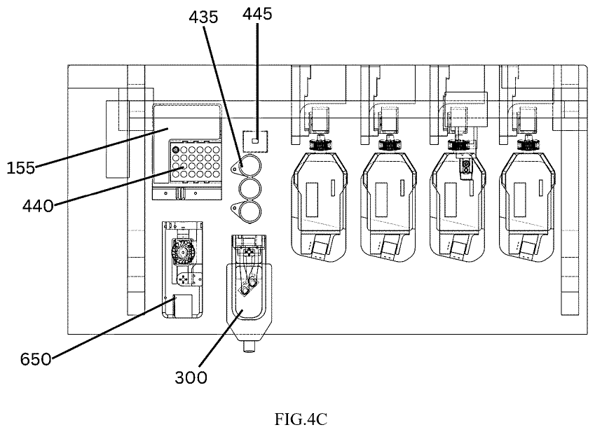

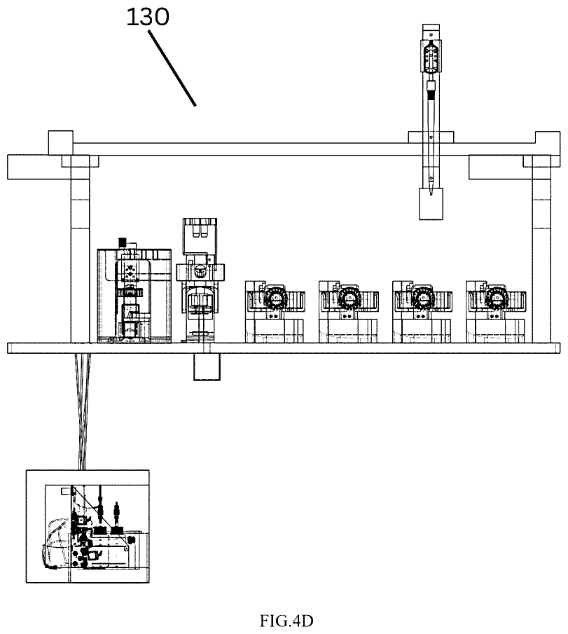

FIG. 3D illustrates a side view of the imaging system (300), wherein the cell culture vessel (140) is statically positioned on the vessel placer (330) for image capture. FIG. 4A displays the interior of the liquid handling compartment. Here, cell culture vessels are brought to the vessel operation platform (145), designed to accommodate various vessel types including, but not limited to, flasks, well plates, multilayer flasks, and petri dishes, adaptable to specific user requirements. The cap opener (135) is employed to remove caps or lids, with its adjustments tailored to each vessel type. Once open, the vessel tilting system (405), gently tilts the vessel, allowing the pipette (410) to perform liquid aspiration and dispensing at the vessel's end without contact with the bottom surface, preserving the cultured and adhered cells. The platform quantity can be scaled up for increased cell culture capacity.

FIG. 4A also illustrates the 3-axis gantry system, including a side gantry system (430) that allows the automatic liquid pipettor (125) to move front and back, a vertical gantry (425) that enables the automatic liquid pipettor (125) to move up and down, and the horizontal gantry (420) that permits the automatic liquid pipettor (125) to move left and right. The movement of the automatic liquid pipettor (125) into different angles is controlled by the rotor (415). The pipetting system inserts the pipette tip into the flask at an angle, facilitated by the rotor (415).

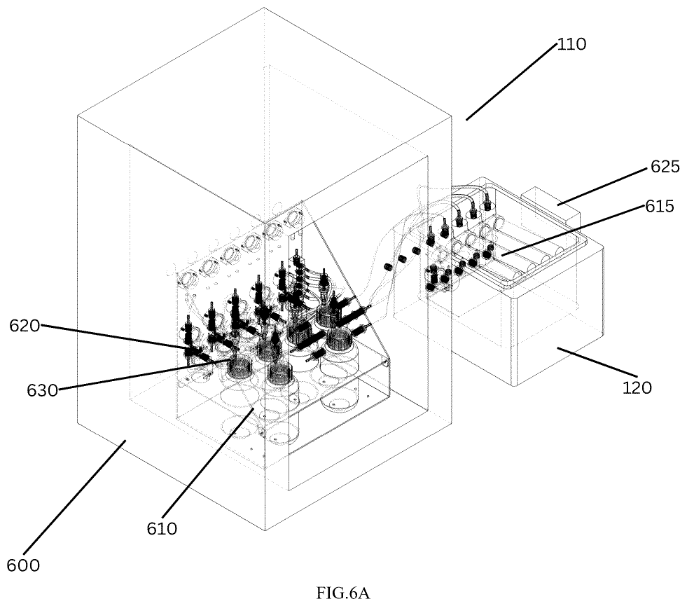

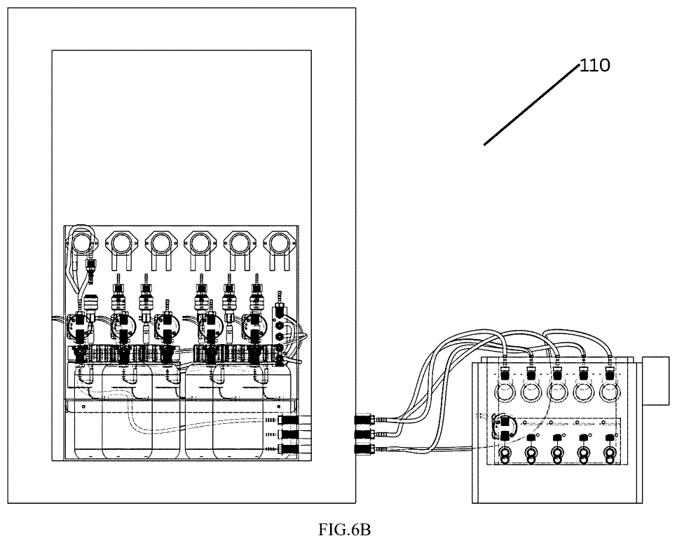

In one embodiment, shown in FIG. 2B, the Liquid Handling Robotic System (130) is configured as a standalone module separable from the full system (100). This standalone system is designed to be installed within a commercial biosafety cabinet or laminar flow hood (200), ensuring that cell culture operations occur in a sterile, enclosed environment. The liquid supply system (110) can be positioned near the liquid handling robotic system (130) for efficient liquid storage, warming, and dispensing as required for cell culture. A detailed visualization of this configuration is illustrated in FIG. 6A.

In another embodiment, depicted in FIG. 2C, the Liquid Handling Robotic System (130) is fully integrated within an enclosed system (205) that features a laminar flow environment. In this setup, the liquid handling compartment (105) is positioned atop an incubation system (210) that includes an automatic door mechanism, which opens and closes to maintain a controlled environment while allowing vessel access. A SCARA (Selective Compliance Articulated Robot Arm) (215) is employed to coordinate and transport cell culture vessels-such as tissue flasks, petri dishes, well plates, and conical tubes-between the Liquid Handling Compartment (105) and the Incubation Compartment (190). The AI-driven system software precisely manages and coordinates the transportation process, optimizing workflow efficiency.

In an alternative embodiment, shown in FIG. 2D, the full system (100) is configured such that the liquid handling compartment (105) is directly mounted atop the automated cell culture incubation compartment (190), reducing overall system footprint while maintaining full automation and sterility.

The incubation compartment (190) is a key component of full system (100), designed to maintain optimal environmental conditions for cell culture growth. The incubation compartment ensures precise control over the cell culture environment by maintaining a constant temperature of (37° C.±0.5° C.), a CO₂ concentration of 5%, and a relative humidity of approximately 95%. This controlled environment prevents desiccation of culture media while supporting proper cell proliferation.

Inside the incubation compartment (190), a rotating shelf (160) is used to store multiple cell culture vessels, including but not limited to flasks, well plates, and petri dishes. The number of racks on the rotating shelf is adjustable, allowing customization based on the number and type of cell culture vessels used in a given experiment. The shelf rotates automatically to position the appropriate vessel for retrieval by the transportation system (165).

A gripper (175), installed on the transportation system (165), functions to retrieve and transport cell culture vessels from the incubation compartment to the liquid handling compartment (105) when liquid handling operations are required. The gripper (175) securely grasps cell culture vessels and works in conjunction with a horizontal and vertical gantry system to ensure precise positioning during transport.

### I. AUTOMATED INCUBATION SYSTEM WITH SCARA INTEGRATION

In one embodiment (FIG. 2C and FIG. 2D), the incubation compartment (190) may be implemented as an automated incubation system (210) that features an automatic door mechanism. This door mechanism automatically opens and closes to facilitate seamless access for a SCARA (Selective Compliance Articulated Robot Arm) (215). The SCARA arm is configured to retrieve, transport, and position cell culture vessels (140) within the incubation and liquid handling compartments, ensuring minimal manual intervention while maintaining sterility. The automation of the door mechanism allows for precise and efficient movement of vessels between compartments while preserving the controlled environment.

### II. AI-DRIVEN IMAGING AND AUTOMATED TRANSPORTATION

In some embodiments, the incubation compartment (190) is equipped with an automated imaging system (300) that periodically captures images of each cell culture vessel to assess cell growth conditions. The imaging system operates in conjunction with an AI model trained on a dataset of cell morphology, confluency levels, and media color changes. Based on real-time image analysis, the AI model determines whether a vessel should be moved to the liquid handling compartment for media exchange or cell passaging.

If AI analysis detects that confluency has reached 80%-90%, the system triggers the transportation system (165) or SCARA arm (215) to retrieve the vessel for passaging. Similarly, if the media color changes due to phenol red pH shifts, indicating waste accumulation, the system initiates an automatic media exchange protocol. If no action is required, the culture vessel remains in the incubation compartment for continued growth.

To enhance automation, the transportation system (165) may also feature a motorized track and sensor feedback mechanisms that ensure smooth, precise movement of vessels between compartments. The AI software optimizes transport efficiency by scheduling vessel retrievals based on the system's overall workload, preventing bottlenecks and improving throughput.

The Liquid Handling Compartment (105) serves as the central processing unit for executing automated liquid handling tasks within the full system (100). It houses multiple robotic subsystems responsible for precise reagent dispensing, media exchange, and cell passaging, ensuring high-throughput and contamination-free cell culture operations.

The compartment is designed as a sterile, enclosed environment, preventing contamination and supporting reproducibility in cell culture protocols. It accommodates a wide range of cell culture vessels, including but not limited to cell culture flasks, petri dishes, well plates, and multilayer culture flasks. The system is adaptable for both adherent and suspension cultures, with automated adjustments based on vessel type and cell growth conditions.

### A. Automatic Liquid Pipettor (125) and Pipetting Operations

The Automatic Liquid Pipettor (125) is a core component of the Liquid Handling Robotic System (130) as shown in FIG. 4A and FIG. 4B, designed for high-precision liquid handling. It is mounted on a multi-axis robotic gantry system, allowing it to move across the workspace to access different cell culture vessels. Key features include:
a. Pipetting Precision: The pipettor ensures accurate liquid transfer using pre-calibrated volume control mechanisms, with adjustments for viscosity and fluid dynamics of different reagents.
b. Disposable Pipette Tip Replacement: To prevent cross-contamination, the system automatically replaces pipette tips after each liquid transfer operation. A Pipette Holder (440) stores fresh tips, while used tips are discarded into a designated waste storage (155).
c. Automated Dispensing: The system supports variable-speed pipetting, enabling controlled aspiration and dispensing of media, trypsin, and buffers without disturbing adherent cells.

### B. Vessel Operation Platform (145) and Cap Handling

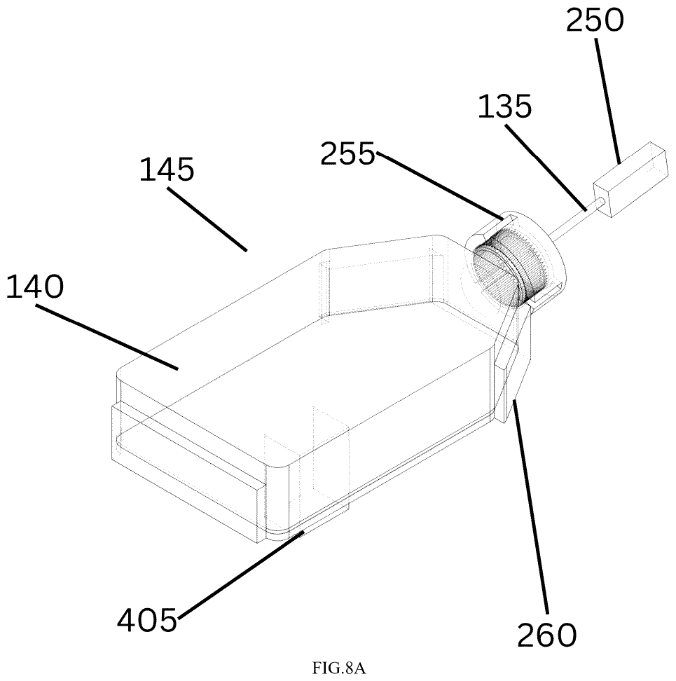

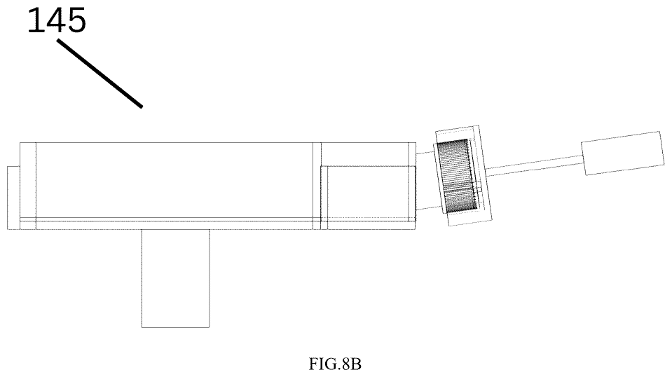

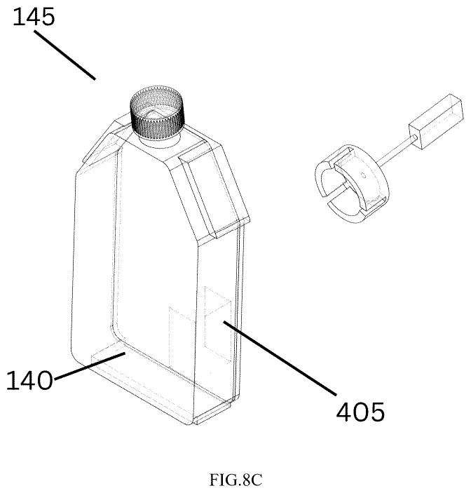

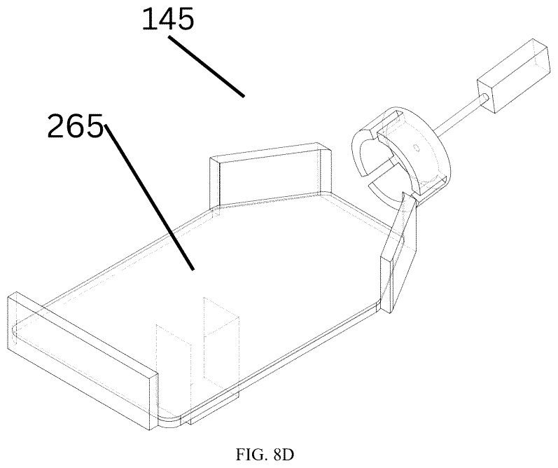

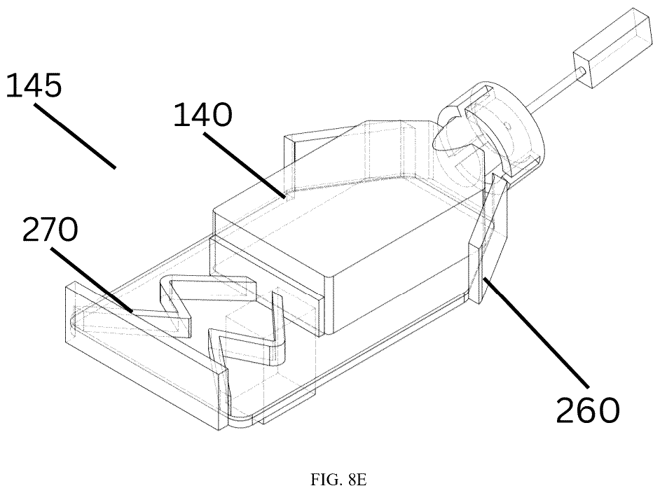

The Vessel Operation Platform (145) as depicted in FIG. 8A is designed to securely hold cell culture vessels during liquid handling. It incorporates:
a. A Cap Opener (135): A linear actuator (250) drives the cap adapter (255), which securely grips and removes vessel caps before liquid handling operations. The cap adapter is adjustable to accommodate different vessel cap sizes and threading mechanisms.
b. A Vessel Tilting System (405): During media exchange and cell passaging, the system tilts the vessel to an optimal angle, preventing liquid spills while allowing precise pipetting.
c. A Heating Module (265) as depicted in FIG. 8D: The platform includes an integrated heating system, ensuring vessels are maintained at an optimal temperature (37° C.±0.5° C.) during liquid handling, preventing cell shock due to sudden temperature changes. The vessel operation platform (145) is modular and can be installed and uninstalled. There are two installed in the embodiment shown in FIG. 4A and four installed in the embodiment shown in FIG. 4B. The number of vessel operation platform (145) being placed on the liquid handling robotic system (130) can be determined based on user's need, and it can be increased to more number based on the customized size of the liquid handling robotic system (130).

FIG. 8B depicts the side view of the vessel operation platform (145).

FIG. 8C depicts how the tilting system worked. The cell culture vessel (140) can be tilted upward, allowing the pipette (410) to be inserted through the opening for liquid aspiration and dispensing.

FIG. 8E illustrates an optional add-on module comprising a vessel adapter (270) configured to enhance the compatibility of the vessel operation platform (145) with smaller-sized cell culture vessels (140). The vessel adapter (270) employs a spring mechanism to urge the smaller-sized cell culture vessel (140) toward an upper edge of the vessel holder (260), thereby securing the vessel in position for liquid handling operations.

### C. Alternative Configurations of the Liquid Handling Robotic System (130)

The Liquid Handling Robotic System (130) is designed to accommodate different laboratory setups and operational needs, with the following configurations:
a. Standalone Configuration (FIG. 2B): The liquid handling robotic system (130) is installed within a commercial biosafety cabinet or laminar flow hood (200), ensuring a sterile, enclosed workspace. The Liquid Supply System (110) is positioned nearby for reagent storage and temperature-controlled dispensing.
b. Integrated Laminar Flow Configuration (FIG. 2C): The liquid handling compartment (105) is positioned atop the incubation system (210), featuring an automatic door mechanism that allows for seamless transport of culture vessels by a SCARA (Selective Compliance Articulated Robot Arm) (215). The entire system maintains a sterile laminar flow environment.
c. Direct Integration Configuration (FIG. 2D): The Liquid Handling Compartment (105) is mounted directly on top of the Incubation Compartment (190), minimizing footprint and improving workflow efficiency in constrained laboratory environments.

Referring to FIG. 2B, the Liquid Handling Robotic System (130) is shown installed within a commercial biosafety cabinet or laminar flow hood (200). This configuration is further illustrated in FIG. 4B, where a liquid supply system (110) is positioned below the Liquid Handling Robotic System (130). The Vessel Operation Platform (145) is mounted on the platform of the Liquid Handling Robotic System (130) and is designed to hold and manipulate cell culture vessels (140). Specifically, it tilts the vessel to facilitate the insertion of a pipette for liquid handling operations. Detailed views of the cell culture vessel (140) being held and operated by the Vessel Operation Platform (145) are provided in FIGS. 8A, 8B, and 8C.

Similar to the alternative embodiment depicted in FIG. 4A, the Liquid Handling Robotic System (130) in FIG. 4B is constructed with a gantry system comprising a Side Gantry (430), a Horizontal Gantry (420), and a Linear Actuator (250). The Linear Actuator (250) is equipped with an Automatic Liquid Pipettor (125), which holds a pipette (410) for liquid handling tasks. The Linear Actuator (250) moves horizontally along the Horizontal Gantry (420), while the Automatic Liquid Pipettor (125) can be adjusted vertically by the Linear Actuator (250). Additionally, the Horizontal Gantry (420) can move forward and backward along the Side Gantry (430), enabling the Automatic Liquid Pipettor (125) to access any location on the Liquid Handling Robotic System (130) for operational purposes.

As shown in FIG. 4B, the Pipette Holder (440) is also located on the platform of the Liquid Handling Robotic System (130). The Pipette Holder (440) stores fresh pipettes to ensure sterile liquid handling. The Automatic Liquid Pipettor (125) retrieves fresh pipettes from the Pipette Holder (440) as needed. Positioned behind the Pipette Holder (440) is the Waste Storage (155), designated for disposing of used pipettes. The Automatic Liquid Pipettor (125) moves to the top of the Waste Storage (155) to discard used pipettes. Additionally, the Imaging System (300) is integrated into the platform of the Liquid Handling Robotic System (130), as clearly depicted in FIG. 4C, which provides a top view of the system. The Imaging System (300) captures images of cells within the cell culture vessel (140) to assess parameters such as confluency and morphology. These images are then transmitted to a computer for analysis.

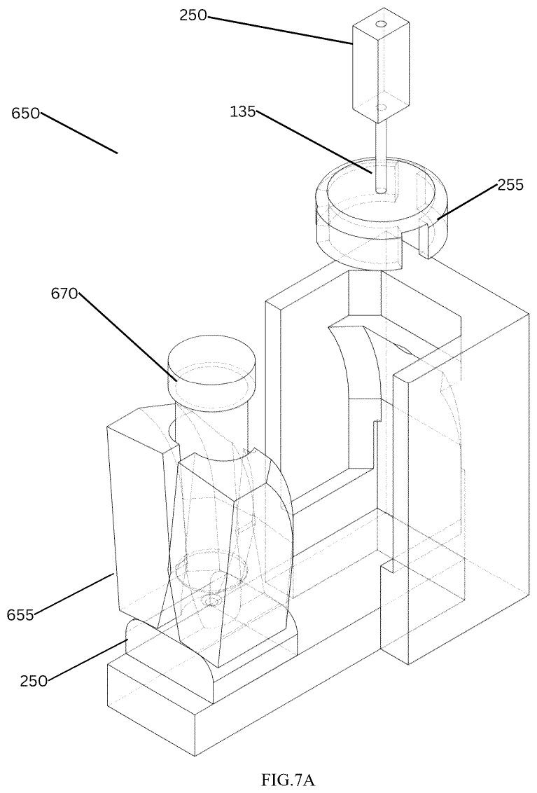

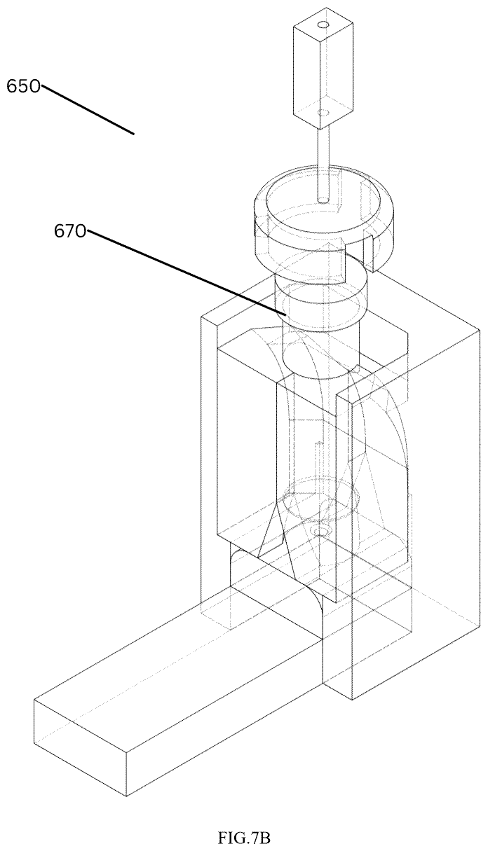

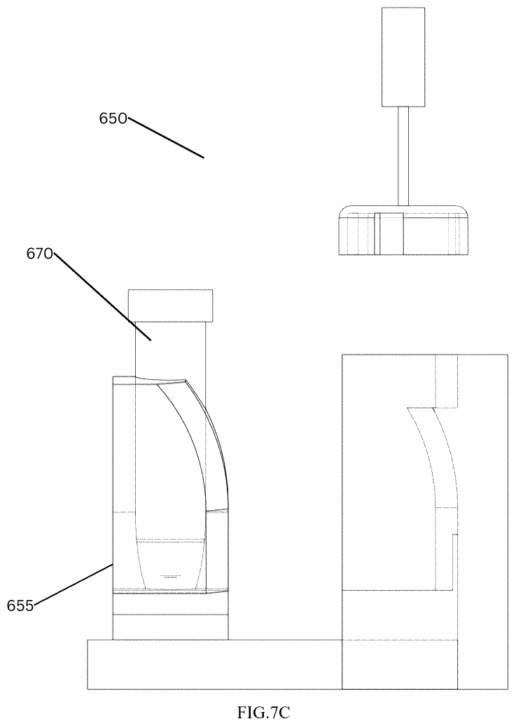

The Conical Tube Operation Platform (650) is also situated on the platform of the Liquid Handling Robotic System (130), visible in both FIG. 4B and FIG. 4C. This platform, detailed in FIGS. 7A, 7B, and 7C, is designed to handle conical tubes (670)—also known as centrifuge tubes. It facilitates the opening and closing of tube caps, enabling the pipetting of liquids into and out of the tubes. Conical tubes are utilized for various purposes, including harvesting cells or media from the cell culture vessel (140) and supplying compounds for cell culture applications.

Referring to FIG. 7A, the Conical Tube Operation Platform (650) is depicted. This platform features a Tube Clamp (655) designed to securely hold conical tubes (670) of various sizes, such as 50 mL and 15 mL. A Linear Actuator (250) moves the Tube Clamp (655) between two positions: an inner position (FIG. 7B) and an outer position (FIG. 7A). In the inner position, a Linear Actuator (250), connected to a Cap Adapter (255), engages the cap of the conical tube by precisely fitting over it. The Cap Adapter (255) grips the cap, allowing the Linear Actuator (250) to move vertically and rotate, thereby loosening and removing the cap to access the tube for liquid handling operations. Once the cap is removed, the Linear Actuator (250) moves the Tube Clamp (655) to the outer position, where liquid addition or removal takes place. FIG. 7C provides a side view of the Conical Tube Operation Platform (650) with the Tube Clamp (655) in the outer position.

Additionally, the system includes a Liquid Reservoir (435) positioned behind the Imaging System (300), as illustrated in FIG. 4C. The Liquid Reservoir (435) stores warmed liquids sourced from the Liquid Supply System (110), where liquids are initially held in a Refrigerator (600), warmed in a liquid heating system (120) and subsequently transferred through a tubing system. FIG. 4D provides a front view of the liquid handling robotic system.

### D. Waste Management Systems

To maintain sterility and reduce cross-contamination risks, the system includes a comprehensive waste management system:
a. Liquid Waste Disposal: A Liquid Waste Container (445) as shown in FIG. 4C collects discarded media, buffers, and trypsin after each liquid handling operation. Waste levels are monitored in real-time, and alerts are generated when disposal is required.
b. Solid Waste Disposal: Used pipette tips are ejected into a Waste Storage 155 as shown in FIGS. 4A, 4B, and 4C. The waste storage capacity is monitored by sensors, prompting replacement when full.
c. Decontamination Protocols: Between operations, the system performs automated rinsing cycles using sterile PBS and ethanol-based cleaning solutions, ensuring minimal contamination risk.

### E. Fluid Storage and Management

The Liquid Supply System (110) as shown in FIG. 6A is responsible for the storage, preparation, and controlled delivery of various liquid reagents required for automated cell culture operations. This system ensures that all necessary fluids—such as cell culture media, phosphate-buffered saline (PBS), trypsin, and washing buffers—are available in a temperature-regulated state, minimizing manual intervention and maintaining consistency in liquid handling procedures.

Referring to FIG. 6A, the Liquid Supply System (110) is illustrated as comprising two primary compartments: Refrigerator (600) and liquid heating system (120)

Refrigerator: Reagents requiring long-term storage are kept in a temperature-controlled environment within the refrigerator (600). This ensures stability and prevents premature degradation of cell culture media and other temperature-sensitive solutions.

Liquid heating system (120) (37° C.): Before dispensing, the selected reagent is transferred to a Liquid Heating System (120), which is equipped with a a temperature control unit (625). The system gradually raises the temperature to 37° C., ensuring that cell culture media and enzymes reach physiological temperature before being dispensed into culture vessels.

Real-Time Temperature Monitoring: A set of temperature sensors continuously tracks the liquid's temperature throughout the heating process, ensuring precise control. If deviations from the target temperature occur, the system automatically adjusts the heating parameters to restore optimal conditions.

Quick Connectors for Fluid Pathways: The quick connectors (630) is designed with quick-connect fittings, allowing easy replacement of reagent lines without contamination risk. This modular design supports rapid reagent switching, reducing downtime when changing between different liquid protocols. The quick connectors (630) enables quick connection between the reagent with the peristaltic pump (620), which pumps the liquid from the refrigerator (600) to the temporary reservoir (615) in the liquid heating system (120)

FIG. 6B depicts a side view of the Liquid Supply System (110).

### III. IMAGING SYSTEM AND AI-POWERED CELL ANALYSIS

### A. Hardware Specifications of the Imaging System

The imaging system (300) as shown in FIG. 3B comprises an inverted microscopic imaging system, designed to capture high-resolution images of adherent and suspension cell cultures. The key components include:

Microscope System (310): Utilizes one or more imaging modalities, including brightfield, phase contrast, and fluorescence microscopy, allowing for comprehensive cell analysis across different experimental conditions. The system can switch between magnifications such as 4×, 10×, 20×, and 40×, supporting detailed morphological assessments.

Autofocus Motor (320): Enables precise focal adjustments to optimize image clarity. The autofocus mechanism is designed to dynamically adjust based on live image feedback, reducing errors in focus drift.

Light Source (305): Provides illumination using adjustable LED or halogen light sources, which can be tuned based on the cell type and imaging requirements.

Camera System (315): Captures images at high resolution, transmitting them to the Microscope Computer (325) for real-time processing. The camera supports time-lapse imaging to monitor cell growth trends over extended culture periods.

### B. AI-Powered Cell Analysis

The imaging system is integrated with an AI-driven analytical module that processes captured images to assess cell health and growth status. The AI module is trained to perform several key functions:

Cell Confluency Detection: The AI model detects and quantifies cell coverage within a culture vessel. If the detected confluency reaches a user-defined threshold (e.g., 80% or 90%), the system initiates an automated cell passaging protocol.

Morphology Assessment: Advanced segmentation algorithms classify individual cells based on size, shape, and clustering patterns to detect abnormalities or contamination. The AI can flag irregular morphologies, prompting alerts for manual review.

Media Condition Monitoring: Utilizes colorimetric analysis of the cell culture media, detecting changes in pH based on phenol red indicator shifts. If the media color suggests nutrient depletion or waste buildup, an automated media change is triggered.

Contamination Detection: Machine learning models trained on contaminated vs. non-contaminated samples analyze images for potential microbial or fungal infections, issuing warnings if contamination is suspected.

### IV. INTEGRATION WITH THE LIQUID HANDLING SYSTEM

The real-time feedback loop between the imaging system and liquid handling system ensures precise culture maintenance:

Automated Pipetting Adjustments: Based on imaging results, the AI determines whether to perform media changes, cell passaging, or additional feeding steps. The liquid handling system then executes the required protocol automatically.

Remote Monitoring and User Interface Integration: Users can access live imaging data via a graphical user interface (GUI), where they can manually adjust imaging parameters, review AI-generated alerts, and override automated decisions if necessary.

Closed-Loop AI Feedback: The AI continuously learns from past imaging data, refining its detection accuracy over time. Adjustments to confluency thresholds, cell morphology assessments, and contamination predictions are made dynamically based on accumulated historical data.

In some embodiments, a computing device may initially analyze the cell images using artificial intelligence, such as that provided in pseudocode algorithms below.

The AI model is trained with cell images of different cell types, cell confluency level, and cell media color with open dataset and dataset we collected with the imaging system (300).

### V. PSEUDOCODE ALGORITHMS

Algorithm 1: MitoAI's AI-driven automated cell culturing system for optimizing cell growth in a lab setting integrates hardware and software to regulate environmental conditions and perform real-time cell monitoring. The AI model analyzes cell health, triggering automated responses for media changes and cell passaging. The system also includes error detection and preventive maintenance protocols, enhancing efficiency and reducing manual intervention in cell culture maintenance.

Algorithm 1: Enhanced AI Manipulated
Automated Cell Culturing System

| Data: | Incubator(105), LiquidHandler(180), |
|---|---|
| | TransportationSystem(165), WasteStorage(155), |
| | ImagingSystem(175), AIModel, UserInterfacePanel(150) |

```
Result: Automated cell culture processing and monitoring
Initialize hardware interfaces: Incubator, LiquidHandler,
  TransportationSystem, WasteStorage, ImagingSystem;
Load AI Model for cell image analysis;
Define image preprocessing steps;
Set environmental parameters: TEMPERATURE_SETPOINT,
  CO2_CONCENTRATION_SETPOINT, HUMIDITY_SETPOINT;
Initialize User Interface Panel for manual protocol settings and
  operations;
Initialize Liquid Supply System(110) with reagent storage and heating
  system(120);
while System. Operational do
```

| \| | Check conditions in Incubator (temperature, CO2, humidity); | | | |
|---|---|---|---|---|
| \| | Adjust conditions to maintain setpoints if necessary; | | | |
| \| | foreach vessel in Incubator do | | | |
| \| | \| | Capture image of cells in vessel using ImagingSystem | | |
| \| | \| | with microscope(310) and light source(305); | | |
| \| | \| | Transmit image to computer(180) for analysis; | | |
| \| | \| | Preprocess image; | | |
| \| | \| | Analyze image using AIModel for cell confluency, | | |
| \| | \| | morphology, and media color; | | |
| \| | \| | if analysis indicates action needed then | | |
| \| | \| | \| | TransportationSystem(165).move(vessel; | |
| \| | \| | \| | Liquid Handler(180)); | |
| \| | \| | \| | LiquidHandler.retrieveReagents(vessel); | |
| \| | \| | \| | LiquidHandler.performTasks(vessel, using pipetting | |
| \| | \| | \| | system(125) and operation platform(145)); | |
| \| | \| | \| | Manage waste using WasteStorage(155) with separate | |
| \| | \| | \| | bins for liquid and solid waste; | |
| \| | \| | \| | if CellPassagingRequired then | |
| \| | \| | \| | \| | Perform cell passaging protocol as described in |
| \| | \| | \| | \| | operation process; |

| \| | \| | \| | end |
|---|---|---|---|
| \| | \| | else | |
| \| | \| | \| | Continue to next vessel; |

| \| | \| | end |
|---|---|---|
| \| | end | |
| \| | if periodic check time then | |
| \| | \| | Check system components for errors or issues; |
| \| | \| | Generate alerts or shutdown if critical issues detected; |

| | |
|---|---|
| \| | end |

end

Algorithm 2: Our Imaging System may process raw images, adjusting image size and color parameters before normalization. The AI Model may take in images captured by the imaging system to determine cell culture conditions. Based on the analysis, the system may decide whether to continue culturing, change media, or perform cell passaging, with tasks executed by an integrated liquid handling robotic system. No action may be taken if the cell culture conditions are satisfactory, allowing the culture to remain in the incubator.

Algorithm 2: Image Preprocessing and AI
Model Analysis for Determining Action

Data: RawImage from ImagingSystem (175)
Result: Determined action based on AIModel analysis
Input: RawImage

| Output: | Determined Action (No action, Media |
|---|---|
| | Change, Cell Passaging) |

| Initialize image transformation parameters: RESIZE_DIMENSIONS, | | | |
|---|---|---|---|
| NORMALIZATION_MEAN, NORMALIZATION_STD; | | | |
| /* | Resize the image | | */ |
| ResizedImage ← Resize(RawImage, RESIZE_DIMENSIONS); | | | |
| /* | Convert image to grayscale if necessary | | */ |
| if Rawimage is colored then | | | |
| \| | GrayImage ← ConvertToGrayscale(ResizedImage); | | |
| else | | | |
| \| | GrayImage ← ResizedImage; | | |
| end | | | |
| /* | Apply normalization | | */ |
| NormalizedImage ← Normalize(GrayImage, | | | |
| NORMALIZATION_MEAN, NORMALIZATION_STD); | | | |
| /* | Additional preprocessing steps can be added here if | | |
| | necessary | | */ |
| /* | Prepare image for AI model | | */ |
| PreprocessedImage ← PrepareForModel(NormalizedImage); | | | |
| /* | Transmit preprocessed image to the computer (180) for | | |
| | AI Model Analysis | | */ |
| AIAnalysisResult ← AIModel(PreprocessedImage); | | | |
| /* | Determine action based on AI analysis | | */ |
| if AIAnalysisResult indicates action required (Media Change, Cell | | | |
| Passaging) then | | | |
| \| | /* | Convey cell culture vessel (140) to liquid | |
| \| | | handling robotic system (130) | */ |
| \| | TransportationSystem(165).move(vessel, | | |
| | LiquidHandler(130)); | | |
| \| | return Action Required: Liquid Handling Task; | | |
| else | | | |
| \| | /* | No action needed, cell culture vessel remains | |
| \| | | in the incubator (160) | */ |

| | |
|---|---|
| \| | return No Action Required: Continue Culturing; |

end

Algorithm 3: Preprocessed images from the Imaging System are passed to the AI Model. The AI Model may determine cell confluency, cell type, morphology, and media color. The AI model, to be loaded with specific parameters and weights, may receive an image and provide outputs which may be further refined through post-processing techniques. The end results may include detailed metrics on cell culture status, and may enable the system to make precise adjustments to the culturing process.

Algorithm 3: AI Model for Predicting Cell Confluency, Classifying
Cell Type, Analyzing Cell Morphology, and Media Color

Data: PreprocessedImage from image preprocessing steps
Result: CellConfluency, CellType, CellMorphology, MediaColor
Load trained AI Model for cell image analysis;
Initialize model parameters and weights;
Input: PreprocessedImage
Output: CellConfluency, CellType, CellMorphology, MediaColor

| /* | Feed preprocessed image into the AI model | */ |
|---|---|---|
| ModelOutput ← AIModel(PreprocessedImage); | | |
| /* | Extract cell confluency prediction | */ |
| CellConfluency ← ExtractConfluency(ModelOutput); | | |
| /* | Extract cell type classification | */ |
| CellType ← ClassifyCellType(ModelOutput); | | |
| /* | Extract cell morphology | */ |
| CellMorphology ← ExtractMorphology(ModelOutput); | | |
| /* | Analyze media color | */ |
| MediaColor ← AnalyzeMediaColor(ModelOutput); | | |
| /* | Post-processing if necessary (e.g., thresholding, | |
| | smoothing) | */ |

PostProcessedConfluency ← PostProcessConfluency(CellConfluency);
PostProcessedCellType ← PostProcessCellType(CellType);
PostProcessedMorphology ← PostProcessMorphology(CellMorphology);
PostProcessedMediaColor ← PostProcessMediaColor(MediaColor);
return PostProcessedConfluency, PostProcessedCellType,

PostProcessedMorphology, PostProcessedMediaColor;

Algorithm 4: The Liquid Supply System is designed for cell culture reagents management. It initiates by preparing the system with reagents at a controlled temperature and then, upon request, delivers the reagents through a tubing system to a heating module. The reagents are warmed to the precise temperature required for cell culture activities and maintained in a heated state until deployment by a robotic system.

Algorithm 4: Liquid Supply System for Cell Culture Reagents

```
Data: Request for Cell Culture Reagents
Result: Delivered and Heated Reagents for Cell Culture Use
Initialize Liquid Supply System with storage containers for reagents
  (e.g., cell culture media, trypsin, PBS buffer);
Initialize Liquid Heating System with separate channels for each
  reagent;
Set temperature of Liquid Supply System to 4°C (like a mini-fridge);
```

| /* | Reagent Delivery Process | | | */ |
|---|---|---|---|---|
| while there is a request for reagents do | | | | |
| \| | foreach requested reagent do | | | |
| \| | \| | /* | Retrieve reagent from storage container | */ |
| \| | \| | Retrieve Reagent from storage container; | | |
| \| | \| | /* | Transport reagent via tubing system to heating | |
| \| | \| | | system | */ |
| \| | \| | Transport Reagent via TubingSystem(115) to | | |
| \| | \| | LiquidHeatingSystem(120); | | |
| \| | \| | /* | Heat reagent to required temperature for cell | |
| \| | \| | | culture use | */ |
| \| | \| | Heat Reagent in LiquidHeatingSystem(120) to 37°C; | | |
| \| | \| | /* | Keep heated reagent in separate channel until used | |
| \| | \| | | by robotic system | */ |

| \| | \| | Maintain Heated Reagent in separate channel ready for use; | |
|---|---|---|---|
| \| | end | | |
| \| | /* | Notify that the reagents are ready for the robotic | |
| \| | | system | */ |

| | |
|---|---|
| \| | Notify Robotic System that reagents are heated and ready for use; |

end

Algorithm 5: The user interaction loop includes updating system status, handling user input and requests, adjusting settings, and processing image data. It also manages user preferences for alerts and error handling, ensuring that any critical issues are communicated to predefined recipients. The system is designed to continuously process and respond to input, maintaining optimal conditions for cell culture growth, and providing a user-friendly interface for system management and monitoring.

Algorithm 5: User Interface for AI Manipulated Automated Cell Culturing System

| Data: | User Inputs, System Status, Cell Images, Predicted Cell Growth |
|---|---|
| | Information |

| | |
|---|---|
| Result: | Processed User Requests, Displayed System Information, |

Stored and Retrieved Cell Images, Displayed Growth
Predictions

| | | |
|---|---|---|
| /* | Initialization of the User Interface | */ |

Initialize User Interface Components: Display, Input Controls, System
Status Indicators, Cell Line Selection, Image Gallery, Growth

Prediction Display;
Load User Preferences and Settings;

Connect to System Backend (Incubator, LiquidHandler, etc.);

| Initialize Cell Line Database and Image Storage; | | | | | | | |
|---|---|---|---|---|---|---|---|
| /* | Main Interaction Loop | | | | | | */ |
| while UI is Active do | | | | | | | |
| \| | /* | Display System Status | | | | | */ |
| \| | Get current status from System Backend; | | | | | | |
| \| | Update System Status Indicators on UI; | | | | | | |
| \| | /* | User Input Handling | | | | | */ |
| \| | if User Input detected then | | | | | | |
| \| | \| | switch Input Type do | | | | | |
| \| | \| | \| | case Command (e.g., Start, Stop, Pause) do | | | | |
| \| | \| | \| | \| | Send command to System Backend; | | | |
| \| | \| | \| | \| | Display command confirmation; | | | |
| \| | \| | \| | end case | | | | |
| \| | \| | \| | case Setting adjustment (e.g., temperature, CO2 level) do | | | | |
| \| | \| | \| | \| | Validate setting adjustment; | | | |
| \| | \| | \| | \| | Send adjustment to System Backend; | | | |
| \| | \| | \| | \| | Display adjustment confirmation; | | | |
| \| | \| | \| | end case | | | | |
| \| | \| | \| | case Cell Line Selection do | | | | |
| \| | \| | \| | \| | Display Cell Line Selection Menu; | | | |
| \| | \| | \| | \| | if Selection confirmed then | | | |
| \| | \| | \| | \| | \| | Send Cell Line Information to System Backend; | | |
| \| | \| | \| | \| | \| | Display confirmation of Cell Line Selection; | | |
| \| | \| | \| | \| | end if | | | |
| \| | \| | \| | end case | | | | |
| \| | \| | \| | case Cell Image Request do | | | | |
| \| | \| | \| | \| | if Request is for saving image then | | | |
| \| | \| | \| | \| | \| | Save current cell image with vessel number and | | |
| \| | \| | \| | \| | \| | timestamp; | | |
| \| | \| | \| | \| | \| | Display confirmation of image saved; | | |
| \| | \| | \| | \| | else | | | |
| \| | \| | \| | \| | \| | if Request is for viewing image then | | |
| \| | \| | \| | \| | \| | \| | Retrieve and display cell images for specified vessel | |
| \| | \| | \| | \| | \| | \| | number; | |
| \| | \| | \| | \| | \| | else | | |
| \| | \| | \| | \| | \| | \| | Display input error message; | |

| | | | | | |
|---|---|---|---|---|---|
| \| | \| | \| | \| | \| | end if |

| \| | \| | \| | \| | end if |
|---|---|---|---|---|
| \| | \| | \| | end case | |
| \| | \| | \| | case Growth Prediction Request do | |
| \| | \| | \| | \| | Retrieve and display predicted cell growth situation; |
| \| | \| | \| | end case | |
| \| | \| | \| | case Health and Growth Report Request do | |
| \| | \| | \| | \| | Generate Report on Cell Health and Growth from System |
| \| | \| | \| | \| | Backend Data; |
| \| | \| | \| | \| | Display and Save Report; |
| \| | \| | \| | end case | |
| \| | \| | \| | case Email Alert Setup do | |
| \| | \| | \| | \| | Configure Email Alert Preferences (recipients, frequency, |
| \| | \| | \| | \| | conditions); |
| \| | \| | \| | \| | Display confirmation of Email Alert Setup; |
| \| | \| | \| | end case | |
| \| | \| | \| | otherwise do | |
| \| | \| | \| | \| | Display input error message; |

| | | | |
|---|---|---|---|
| \| | \| | \| | end case |

| | | |
|---|---|---|
| \| | \| | end switch |

| \| | end if | | |
|---|---|---|---|
| /* | Alert and Error Handling | */ | end while |

| if Alert or Error received from System Backend then | | | |
|---|---|---|---|
| \| | Display Alert or Error message; | | |
| \| | Provide troubleshooting options if applicable; | | |
| \| | if Alert is Critical then | | |
| \| | \| | Send Email Alert to configured recipients; | |
| \| | end if | | |
| end if | | | |
| /* | Periodic Health and Growth Monitoring | | */ |
| if time for periodic monitoring then | | | |
| \| | Get Cell Health and Growth Data from System Backend; | | |
| \| | Analyze Data for any anomalies or critical conditions; | | |
| \| | if Critical conditions detected then | | |
| \| | \| | Send Email Alert with Health and Growth Report; | |

| | |
|---|---|
| \| | end if |
| \| | Generate and Save Periodic Health and Growth Report; |

end if

### VI. CELL CULTURE PROCESS

FIG. 3A demonstrates the cell culture process. In some embodiments which utilize artificial intelligence, a machine learning model may assess cell confluency, morphology, and media color to determine if the cells require media change or cell passaging. Cell passage, also known as subculturing, involves transferring cells from a crowded culture vessel to a new vessel to provide more space for continued growth. This process may be useful in cell culture to maintain healthy cells, prevent overgrowth, and ensure that cells have adequate nutrients and space to proliferate. The procedure typically includes detaching cells from the original culture dish or flask (if they are adherent cells), counting the cells, and seeding them into a new culture vessel at a lower density to allow room for growth. The AI may analyze the image taken from the imaging system (300) on the cell confluency, checking if the cells are reaching to the overgrown stage or not. FIG. 3C demonstrates the different cell confluency level under microscopic imaging. If not overgrown, then no action is needed, the imaging system proceeds to the next vessel for image capture and analysis. If the cells are overgrown, for example 80% or 90% confluency, then an action is required, like cell passaging, the device controls the transportation system to move the vessel to the liquid handling compartment. If the media color has changed, due to the phenol red changing color in the media as an indicator of the waste level in the media, the AI system may also trigger robotic action for media changing.

Upon deciding to take action based on the cell image analysis, the transportation system (165) transfers the cell culture vessel from the incubator to the liquid handling system. This system includes a gripper (175) for handling the vessels, a horizontal gantry for moving the gripper (175) front and back, and a vertical gantry for lifting and lowering the gripper (175). The transportation system and gripper are demonstrated in FIG. 1B.

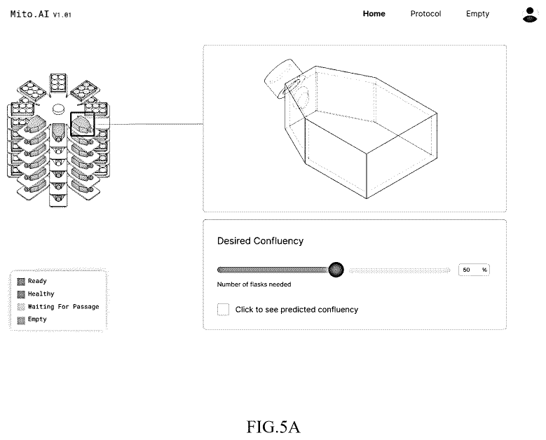

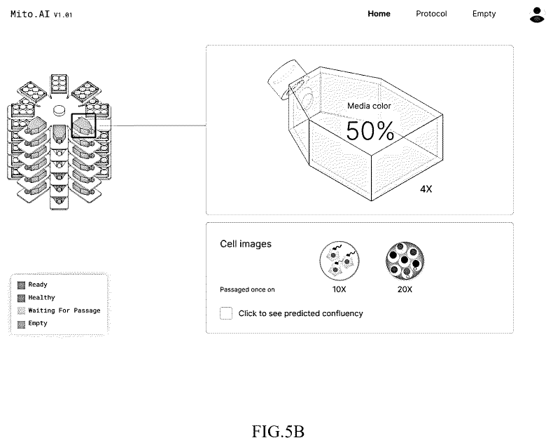

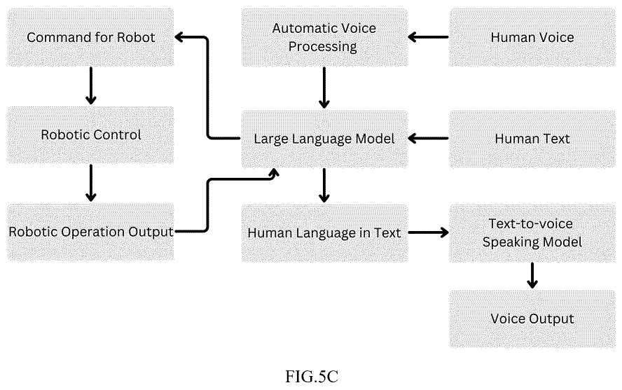

The user interface panel (150) as shown in FIG. 1B enables the user to manually set protocols and operate the liquid handling system. FIG. 5A mad FIG. 5B are examples of the user interface. The user interface allows user to visualize cells in at least one of the magnifications 2×, 4×, 10×, 20×, and 40×. The user interface allows users to operate the system by setting up protocols based on user's need. The user interface allow users to remotely monitor the full cell culture process and remotely control and operate the system. The user interface allows user to receive update on the status of the cell culture process.

The user interface is enhanced by an AI-powered control system, as illustrated in FIG. 5C. This system enables a user to issue verbal commands to the robot in English or other languages. These voice inputs are processed by an automatic voice processing model, which converts them into text. The automatic voice processing model may be either an open-source implementation or developed in-house. The resulting text is transmitted to a large language model, which may be an open-source model or accessed via a commercially available third-party application programming interface (API). The large language model processes the text into robotic commands, which are then relayed to the robotic control system to operate the full system (100). Upon executing the protocols or actions specified by the user, the system generates operational outputs, which are sent back to the large language model for conversion into human-readable text. This text can be displayed to the user, transmitted to the user, or further processed by a text-to-voice speaking model—either developed in-house or sourced from an open-source model—to generate a voice output, effectively enabling the robot to communicate responses back to the user. Below is a step by step cell passaging process conducted using the full system (100) as depicted in FIG. 1A as well as alternative embodiments shown in FIGS. 2B, 2C, and 2D.

The full system (100) includes an AI-driven automated cell passaging process to ensure optimal cell culture conditions while minimizing manual intervention. The process begins by detecting when cells have reached a confluency threshold, typically 80% or 90%, which is determined by the imaging system (300). Once the threshold is met, the system executes an automated passage cycle as follows:
a. Step 1: Media Removal
   i. A pipette (410) equipped with a disposable tip removes the existing culture media from Flask 1, ensuring minimal cross-contamination.
   ii. The used tip is discarded into the waste storage (155).
b. Step 2: PBS Rinse
   i. A fresh pipette tip (tip 2) is used to dispense PBS buffer into Flask 1 for rinsing, removing residual media and debris.
   ii. PBS is aspirated, and the pipette tip is discarded.
c. Step 3: Trypsinization
   i. A new pipette tip (tip 3) transfers trypsin from the liquid supply system (110) into Flask 1 to initiate cell detachment.
   ii. The flask is heated at 37° C. on the vessel operation platform (145) for a predefined duration (e.g., 1-3 minutes).
d. Step 4: Imaging and AI Analysis
   i. After heating at 37° C. for a predefined duration, the flask is moved to the imaging system
   ii. The imaging system (300) analyzes cell detachment based on morphological changes (flattened-to-rounded transition).
   iii. If detachment is incomplete, Flask 1 is returned to the incubator for additional time or subjected to mechanical vibration on the liquid handling platform.
e. Step 5: Neutralization and Transfer
   i. Once cells detach, a fresh pipette tip (tip 4) dispenses culture media to neutralize trypsin.
   ii. Half of the detached cell solution is transferred to a new flask (Flask 2) for further culture expansion.
f. Step 6: Final Media Addition and Incubation
   i. A final pipette tip (tip 6) dispenses fresh media into both flasks to achieve the working volume.
   ii. The flasks are transported back to the incubation system (210), and the automated door mechanism closes, maintaining sterility.

The full system (100) is designed to support multi-passage cultures by leveraging AI-driven scheduling and automation:
a. Confluency-Based Scheduling: The AI continuously monitors confluency levels, ensuring timely passaging to maintain optimal cell growth conditions.
b. Automated Handling of Successive Passages: The system tracks each passage event, logging vessel history and ensuring sequential transfer of cell populations to maintain lineage integrity.
c. Scalability for Bulk Cell Production: The liquid handling system (130) is optimized for multi-vessel processing, allowing simultaneous passaging of multiple cell culture vessels.

### User Interaction and Override Mechanisms

Manual Override via User Interface (FIGS. 5A & 5B): Operators can modify or delay passage schedules through an intuitive graphical user interface (GUI).

Customizable Passage Protocols: Users can define specific enzyme concentrations, incubation times, and passage intervals, adapting the system for various cell lines.

AI-Guided Recommendations: The system provides real-time alerts and suggested modifications for optimized passage timing based on accumulated data.

### VII. EXAMPLES OF COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A cell culture automation system comprising: an artificial intelligence (AI) module configured to analyze cell cultures and make decisions based on said analysis; and a robotic liquid handling system operatively connected to the AI module, the robotic liquid handling system being configured to execute tasks based on the decisions made by the AI module.

### Example 2

The cell culture automation system of any of the previous examples, wherein the robotic liquid handling system is configured as a standalone module adapted for installation within a commercial biosafety cabinet or laminar flow hood and is configured for use with at least one of a tissue flask, petri dish, well plate, and conical tube.

### Example 3

The cell culture automation system of any of the previous examples, wherein the robotic liquid handling system is integrated into an enclosed system comprising a liquid handling compartment positioned atop an incubation system that features an automatic door mechanism, and further includes a Selective Compliance Articulated Robot Arm (SCARA) configured to coordinate and transport cell culture vessels between the liquid handling compartment and the incubation system.

### Example 4

The cell culture automation system of any of the previous examples, wherein the robotic liquid handling system is equipped with a pipetting system compatible with either commercially available serological pipette tips or in-house developed large-volume pipette tips.

### Example 5

The cell culture automation system of any of the previous examples, wherein the enclosed system is ventilated using a High-Efficiency Particulate Air (HEPA) filter filtration system to provide laminar flow, maintaining a sterile internal environment with filtered air.

### Example 6

The cell culture automation system of any of the previous examples, wherein the enclosed system is configured to function as a biosafety cabinet, enabling a user to open and close a door to access and place consumables, reagents, cell culture vessels, and other materials within the enclosed system.

### Example 7

The cell culture automation system of any of the previous examples, further comprising an inverted microscopy imaging system configured to capture images of the cell cultures, the inverted microscopy imaging system including: one or more light sources; a microscope system with multiple magnification lenses; an autofocus mechanism; and a camera, wherein the captured images are transmitted to a microscope computer and subsequently to a main computer for processing, analysis, and decision-making.

### Example 8

The cell culture automation system of any of the previous examples, further comprising a cell culture incubation compartment that includes a digital imager configured to capture images of cells through a cell culture vessel, wherein the cell culture vessel remains within the incubation compartment during imaging without requiring transportation out of the incubation compartment.

### Example 9

The cell culture automation system of any of the previous examples, further comprising a vessel operation platform, wherein the vessel operation platform comprises: a cap opener driven by a linear actuator that reciprocates a cap adapter to engage and remove a cap of a cell culture vessel; a vessel holder configured to securely accommodate the cell culture vessel; a heating module integrated into the vessel holder to maintain the cell culture vessel at an optimal temperature during liquid handling; and a vessel tilting system configured to orient the cell culture vessel upward, thereby facilitating liquid removal or addition by a pipette.

### Example 10

The cell culture automation system of any of the previous examples, further comprising a conical tube operation platform configured to securely hold conical tubes, the conical tube operation platform including: a motor configured to rotate a conical tube adapter between an inner position and an outer position, wherein the inner position includes a linear actuator and a cap adapter configured to engage and remove a cap of a conical tube, and the outer position is configured to enable liquid handling operations.

### Example 11

The cell culture automation system of any of the previous examples, further comprising an automated incubation compartment configured to maintain optimal cell culture conditions, including a temperature of approximately 37° C. (±0.5° C.), a CO₂ concentration of approximately 5%, and a humidity of approximately 95%, the automated incubation compartment further comprising: a rotating shelf with adjustable racks configured to hold cell culture vessels; and a transportation system with a gripper configured to automatically transfer the cell culture vessels from the automated incubation compartment to an imaging system for analysis.

### Example 12

The cell culture automation system of any of the previous examples, wherein the AI module incorporates machine learning algorithms trained on datasets comprising cell culture parameters and outcomes, the AI module being configured to automate decision-making processes for various cell types and culture conditions.

### Example 13

The cell culture automation system of any of the previous examples, further comprising a user interface configured to enable a user to input specific experimental protocols, monitor a status of a cell culture process, and remotely operate the cell culture automation system.

### Example 14

A cell culture automation system comprising: a large language model configured to process human language into robotic commands for controlling a robot and executing protocols; an automatic voice processing system configured to detect and process human voice, convert the human voice into text, and input the text into the large language model for further processing; a speaking model configured to process text output from the large language model into voice to communicate updates and information to a user.

### Example 15

The cell culture automation system of example 14, wherein the large language model is incorporated into an AI module and is configured to process human requests or questions in text form and convert the human language into commands for robotic control, the large language model being either an open-source model or accessed via a third-party commercially available application programming interface (API).

### Example 16

The cell culture automation system of any of examples 14-15, wherein the automatic voice processing system is incorporated into an AI module and is configured to process human voice, convert the human voice into text, and input the text into the large language model for processing to control a robot and interface with the system, the automatic voice processing system being either developed in-house or adopted from an open-source model.

### Example 17

The cell culture automation system of any of examples 14-16, wherein the speaking model is incorporated into an AI module and is configured to convert text output from the large language model into voice, enabling the robot to communicate messages requested by a user or provide updates, the speaking model being either developed in-house or sourced from an open-source model.

### Example 18

A cell culture automation system comprising: a liquid supplying system configured to store liquids in cold conditions, warm the liquids, and dispense the liquids as required for cell culture automation; a waste storage system configured to store and dispose of liquid waste and solid waste separately.

### Example 19

The cell culture automation system of example 18, wherein the liquid supplying system comprises: a refrigerator configured to store bottles containing liquids including cell culture media, phosphate-buffered saline (PBS), and trypsin; a peristaltic pump configured to transfer the liquids from the refrigerator to a liquid heating system; and a liquid heating system comprising: a water bath compartment with a temperature control unit; a temporary reservoir configured to store warmed liquids; and quick connectors configured for rapid tube replacement.

### Example 20

The cell culture automation system of any of any of examples 18-19, wherein the waste storage system comprises: a liquid waste container configured to collect liquid waste including waste media, PBS, and trypsin; and a solid waste container configured to collect solid waste including used pipettes.

### VIII. MISCELLANEOUS

The cell culture automation system of any of the previous examples, further comprising a user interface configured to allow user to input specific experimental protocols and to receive updates on the status of the cell culture process. The cell culture automation system of any of the previous examples, further comprising a user interface configured to allow users to remotely monitor and to remotely operate the cell culture automation system.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these implementations may be readily apparent to those skilled in the art, and generic principles defined herein may be applied to other implementations. Thus, many changes and modifications may be made to the subject technology, by one having ordinary skill in the art, without departing from the scope of the subject technology. For instance, different numbers of a given module or unit may be employed, a different type or types of a given module or unit may be employed, a given module or unit may be added, or a given module or unit may be omitted.

Some versions of the examples described herein may be implemented using a processor, which may be part of a computer system and communicate with a number of peripheral devices via bus subsystem. Versions of the examples described herein that are implemented using a computer system may be implemented using a general-purpose computer that is programmed to perform the methods described herein. Alternatively, versions of the examples described herein that are implemented using a computer system may be implemented using a specific-purpose computer that is constructed with hardware arranged to perform the methods described herein. Versions of the examples described herein may also be implemented using a combination of at least one general-purpose computer and at least one specific-purpose computer.

In versions implemented using a computer system, each processor may include a central processing unit (CPU) of a computer system, a microprocessor, an application-specific integrated circuit (ASIC), other kinds of hardware components, and combinations thereof. A computer system may include more than one type of processor. The peripheral devices of a computer system may include a storage subsystem including, for example, memory devices and a file storage subsystem, user interface input devices, user interface output devices, and a network interface subsystem. The input and output devices may allow user interaction with the computer system. The network interface subsystem may provide an interface to outside networks, including an interface to corresponding interface devices in other computer systems. User interface input devices may include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system.

In versions implemented using a computer system, a user interface output device may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide a non-visual display such as audio output devices. In general, use of the term “output device” is intended to include all possible types of devices and ways to output information from computer system to the user or to another machine or computer system.

In versions implemented using a computer system, a storage subsystem may store programming and data constructs that provide the functionality of some or all of the modules and methods described herein. These software modules may be generally executed by the processor of the computer system alone or in combination with other processors. Memory used in the storage subsystem may include a number of memories including a main random-access memory (RAM) for storage of instructions and data during program execution and a read only memory (ROM) in which fixed instructions are stored. A file storage subsystem may provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem in the storage subsystem, or in other machines accessible by the processor.

In versions implemented using a computer system, the computer system itself may be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the example of the computer system described herein is intended only as a specific example for purposes of illustrating the technology disclosed. Many other configurations of a computer system are possible having more or fewer components than the computer system described herein.

As an article of manufacture, rather than a method, a non-transitory computer readable medium (CRM) may be loaded with program instructions executable by a processor. The program instructions when executed, implement one or more of the computer-implemented methods described above. Alternatively, the program instructions may be loaded on a non-transitory CRM and, when combined with appropriate hardware, become a component of one or more of the computer-implemented systems that practice the methods disclosed.

Underlined and/or italicized headings and subheadings are used for convenience only, do not limit the subject technology, and are not referred to in connection with the interpretation of the description of the subject technology. All structural and functional equivalents to the elements of the various implementations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

I/We claim:

1. A cell culture automation system comprising:
(a) an artificial intelligence (AI) module configured to analyze cell cultures and make decisions based on said analysis;
(b) a robotic liquid handling system operatively connected to the AI module and configured to execute cell culture tasks based on the decisions made by the AI module.

2. The cell culture automation system of claim 1, wherein the robotic liquid handling system is configured as a standalone module adapted for installation within a commercial biosafety cabinet or laminar flow hood and is configured for use with at least one of a tissue flask, petri dish, well plate, and conical tube.

3. The cell culture automation system of claim 1, wherein the robotic liquid handling system is integrated into an enclosed system comprising a liquid handling compartment positioned atop an incubation system that features an automatic door mechanism, the enclosed system further including a Selective Compliance Articulated Robot Arm (SCARA) configured to coordinate and transport cell culture vessels between the liquid handling compartment and the incubation system.

4. The cell culture automation system of claim 1, wherein the robotic liquid handling system is equipped with a pipetting system compatible with either commercially available serological pipette tips or in-house developed large-volume pipette tips.

5. The cell culture automation system of claim 3, wherein the enclosed system is ventilated using a High-Efficiency Particulate Air (HEPA) filter filtration system to provide laminar flow, maintaining a sterile internal environment with filtered air.

6. The cell culture automation system of claim 3, wherein the enclosed system is configured to function as a biosafety cabinet, enabling a user to open and close a door to access and place consumables, reagents, cell culture vessels, and other materials within the enclosed system.

7. The cell culture automation system of claim 1, further comprising an inverted microscopy imaging system configured to capture images of the cell cultures, the inverted microscopy imaging system including:
(a) one or more light sources;
(b) a microscope system with multiple magnification lenses;
(c) an autofocus mechanism; and
(d) a camera, wherein the captured images are transmitted to a microscope computer and subsequently to a main computer for processing, analysis, and decision-making.

8. The cell culture automation system of claim 1, further comprising a cell culture incubation compartment that includes a digital imager configured to capture images of cells through a cell culture vessel, wherein the cell culture vessel remains within the incubation compartment during imaging without requiring transportation out of the incubation compartment.

9. The cell culture automation system of claim 1, further comprising a vessel operation platform, wherein the vessel operation platform comprises:
(a) a cap opener driven by a linear actuator that reciprocates a cap adapter to engage and remove a cap of a cell culture vessel;
(b) a vessel holder configured to securely accommodate the cell culture vessel;
(c) a heating module integrated into the vessel holder to maintain the cell culture vessel at an optimal temperature during liquid handling; and
(d) a vessel tilting system configured to orient the cell culture vessel upward, thereby facilitating liquid removal or addition by a pipette.

10. The cell culture automation system of claim 1, further comprising a conical tube operation platform configured to securely hold conical tubes, the conical tube operation platform including a motor configured to rotate a conical tube adapter between an inner position and an outer position, wherein the inner position includes a linear actuator and a cap adapter configured to engage and remove a cap of a conical tube; and wherein the outer position is configured to enable liquid handling operations.

11. The cell culture automation system of claim 1, further comprising an automated incubation compartment configured to maintain optimal cell culture conditions, including a temperature of approximately 37° C. (±0.5° C.), a CO₂ concentration of approximately 5%, and a humidity of approximately 95%, the automated incubation compartment further comprising:
(a) a rotating shelf with adjustable racks configured to hold cell culture vessels; and
(b) a transportation system with a gripper configured to automatically transfer the cell culture vessels from the automated incubation compartment to an imaging system for analysis.

12. The cell culture automation system of claim 1, wherein the AI module incorporates machine learning algorithms trained on datasets comprising cell culture parameters and outcomes, the AI module being configured to automate decision-making processes for various cell types and culture conditions.

13. The cell culture automation system of claim 1, further comprising a user interface configured to enable a user to input specific experimental protocols, monitor a status of a cell culture process, and remotely operate the cell culture automation system.

14. A cell culture automation system comprising:
(a) a large language model configured to process human language into robotic commands for controlling a robot and executing protocols;
(b) an automatic voice processing system configured to detect and process human voice, convert the human voice into text, and input the text into the large language model for further processing; and
(c) a speaking model configured to process text output from the large language model into voice to communicate updates and information to a user.

15. The cell culture automation system of claim 14, wherein the large language model is incorporated into an AI module and is configured to process human requests or questions in text form and convert the human language into commands for robotic control, the large language model being either an open-source model or accessed via a third-party commercially available application programming interface (API).

16. The cell culture automation system of claim 14, wherein the automatic voice processing system is incorporated into an AI module and is configured to process human voice, convert the human voice into text, and input the text into the large language model for processing to control a robot and interface with the system, the automatic voice processing system being either developed in-house or adopted from an open-source model.

17. The cell culture automation system of claim 14, wherein the speaking model is incorporated into an AI module and is configured to convert text output from the large language model into voice, enabling the robot to communicate messages requested by a user or provide updates, the speaking model being either developed in-house or sourced from an open-source model.

18. A cell culture automation system of claim 1, further comprising:
(a) a liquid supplying system configured to store liquids in cold conditions, warm the liquids, and dispense the liquids as required for cell culture automation; and
(b) a waste storage system configured to store and dispose of liquid waste and solid waste separately.

19. The cell culture automation system of claim 18, wherein the liquid supplying system comprises:
(a) a refrigerator configured to store bottles containing liquids including cell culture media, phosphate-buffered saline (PBS), and trypsin;
(b) a peristaltic pump configured to transfer the liquids from the refrigerator to a liquid heating system; and
(c) a liquid heating system comprising:
(1) a water bath compartment with a temperature control unit;
(2) a temporary reservoir configured to store warmed liquids; and
(3) quick connectors configured for rapid tube replacement.

20. The cell culture automation system of claim 18, wherein the waste storage system comprises:
(a) a liquid waste container configured to collect liquid waste including waste media, PBS, and trypsin; and
(b) a solid waste container configured to collect solid waste including used pipettes.
